SEQUENCE LISTING (1 sequence)

SEQ ID NO: 1 - protein, 261 aa, unidentified
MAGDSEQTLQNHQQPNGGEPFLIGVSGGTASGKSSVCAKIVQLLGQNEVDYRQKQVVILSQDSFYRVLTSEQKAKALKGQFNFDHPDAFDNELILKTLKEITEGKTVQIPVYDFVSHSRKEETVTVYPADVVLFEGILAFYSQEVRDLFQMKLFVDTDADTRLSRRVLRDISERGRDLEQILSQYITFVKPAFEEFCLPTKKYADVIIPRGADNLVAINLIVQHIQDILNGGPSKRQTNGCLNGYTPSRKRQASESSSRPH

(19) United States
(12) Patent Application Publication
(10) Pub. No.: US 2025/0179478 A1
(43) Pub. Date: Jun. 5, 2025

(54) FLUORESCENT METABOLICALLY INCORPORATED NUCLEOSIDES FOR LIVE CELL IMAGING OF RN

(71) Applicants: The Trustees of Princeton University, Princeton, NJ (US); San Diego State University Research Foundation, San Diego, CA (US)

(72) Inventors: Ralph KLEINER, Princeton, NJ (US); Danyang WANG, Princeton, NJ (US); Byron PURSE, San Diego, CA (US); Ana SHALAMBERIDZE, San Diego, CA (US)

(73) Assignees: The Trustees of Princeton University, Princeton, NJ (US); San Diego State University Research Foundation, San Diego, CA (US)

(21) Appl. No.: 18/842,235

(22) PCT Filed: Feb. 28, 2023
(86) PCT No.: PCT/US2023/014038

§ 371 Date: Aug. 28, 2024

Related U.S. Application Data
(60) Provisional application No. 63/314,885, filed on Feb. 28, 2022.

(51) Int. Cl. C12N 15/10

(52) U.S. Cl. CPC C12N 15/1086; C12N 15/1055

(57) ABSTRACT

Disclosed is a platform for fluorescence imaging of bulk RNA dynamics in living cells. The platform utilizes a technique including exposing a plurality of live cells configured to overexpress a WT or mutant ribonucleoside kinase UCK2 under control of an inducible promoter, a constitutively active promoter, or both, to a first quantity of a fluorescent nucleoside (such as fluorescent bicyclic and tricyclic cytidine analogues) for a first period of time.

### CROSS-REFERENCE TO RELATED APPLICATIONS

This application claims priority to U.S. Provisional Patent Application 63/314,885, filed Feb. 28, 2022, the entire contents of which are incorporated by reference herein.

### STATEMENT REGARDING FEDERALLY SPONSORED RESEARCH OR DEVELOPMENT

This invention was made with government support under Grant #R01-GM132189 awarded by the National Institutes of Health and Grant Nos. MCB-1942565, 1800529 and 2102642 awarded by the National Science Foundation. The government has certain rights in the invention.

### REFERENCE TO A SEQUENCE LISTING

This application contains a Sequence Listing in computer readable form. The computer readable form is incorporated herein by reference.

### TECHNICAL FIELD

This application is drawn to fluorescence imaging, and specifically to a platform for fluorescence imaging of bulk RNA dynamics in living cells using fluorescent metabolically incorporated nucleosides.

### BACKGROUND

RNA plays a central role in biology and probing its dynamic behavior is critical for illuminating the mechanisms underlying fundamental biological processes. A powerful approach to study the lifecycle of cellular RNA, including its transcription, trafficking, and decay is fluorescence microscopy; however live-cell RNA imaging remains a major challenge. Current approaches for visualizing RNA dynamics in living cells have primarily focused on imaging individual transcripts. This can be accomplished in several ways. Singer and co-workers developed MS2 tagging, which uses a viral MS2 stem-loop sequence to recruit a GFP-fusion protein to the RNA of interest, and has been the gold standard for live-cell RNA imaging over the last three decades. More recently, fluorescent RNA aptamers and CRISPR-Cas targeting strategies have provided complementary methods to image individual RNA sequences in cells. Finally, RNAs generated in vitro can be chemoenzymatically modified with synthetic fluorophores and then introduced into cells. These methods for live-cell RNA imaging have provided important biological insights into the behavior of individual RNA transcripts, however, they lack the simplicity of fluorescent protein-fusions, and suffer from various drawbacks including low signal, high background fluorescence, and the necessity to introduce non-native RNA sequences into the transcript of interest. In addition, none of these methods can monitor global RNA dynamics in a living cell.

### BRIEF SUMMARY

Various deficiencies in the prior art are addressed below by the disclosed compositions of matter and techniques.

In one aspect of the present disclosure, a method for incorporating a fluorescent nucleoside into cellular RNA may be provided. The method may include exposing a plurality of live cells (such as, e.g., human cells) configured to overexpress an enzyme, WT or mutant ribonucleoside kinase UCK2 (such as, e.g., a Y65G mutant of UCK2) under control of an inducible promoter, a constitutively active promoter, or both, to a first quantity of a fluorescent nucleoside for a first period of time. The method may also include chemically synthesizing the fluorescent nucleoside. In some embodiments, the fluorescent nucleoside may include an extended ring systems projecting from a non-Watson Crick face of the fluorescent nucleoside. In some embodiments, the fluorescent nucleoside may be a fluorescent bicyclic and tricyclic cytidine analogue. The method may also include washing out free nucleoside after the first period of time. The method may also include inducing the inducible promoter to overexpress the WT or mutant ribonucleoside kinase UCK2, where the inducible promoter may be, e.g., a chemically inducible promoter, such as, e.g., a tetracycline-inducible promoter. The method may also include exposing the plurality of live cells to a second quantity of the fluorescent nucleoside for a second period of time. The method may also include imaging the plurality of live cells using epifluorescence microscopy.

In another aspect, a kit may be provided. The kit may include a plurality of cells configured to overexpress a WT or mutant ribonucleoside kinase UCK2 under control of an inducible promoter, a constitutively active promoter, or both. The kit may include a fluorescent nucleoside. The kit may optionally include a chemical agent adapted to induce the inducible promoter.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart of an embodiment of a method.

FIG. 2 includes chemical structures of some fluorescent nucleosides.

FIGS. 3A and 3B are graphs showing RNA incorporation of tC or pyrroloC into (3A) HeLa cells transfected with UCK2 constructs and (3B) HeLa cell lines stably expressing inducible UCK2 WT or Y65G mutant.

FIG. 4 is a graph showing quantification and kinetic analysis of fluorescent nucleoside phosphorylation reactions.

FIG. 5 is an illustration showing an overlay of simulated pyrroloC and cytidine bound to UCK2.

FIGS. 6A and 6B are graphs showing cell viability under various concentrations of tC (6A) or PyrroloC (6B) treatments.

FIG. 7A is a graph showing fluorescence intensity quantification of cells grown in 5% or 10% FBS or in the presence of ActD. Error bars represent mean±s.d (n=50 cells from three independent biological replicates). ****P<0.0001.

FIG. 7B is a graph showing fluorescence intensity quantification of cells for a time-course analysis of tC incorporation into cellular RNA. Error bars represent mean±s.d. (n=50 cells from three independent biological replicates). *P<0.5, ****P<0.0001.

FIG. 7C is a graph showing fluorescence intensity quantification of cells for a time-course analysis of tC incorporation into cellular RNA during co-treatment with 200 μM NaAsO₂. Error bars represent mean±s.d. (n=50 cells from three independent biological replicates). **P<0.01, ****P<0.0001.

FIG. 7D is a graph showing fluorescence intensity quantification of cells pulsed with tC for 4 hours and chased with complete medium or with 200 μM NaAsO₂. Error bars represent mean±s.d. (n=25 cells from three independent biological replicates). **P<0.01, ****P<0.0001.

FIG. 7E is a graph showing quantification of cellular RNA foci number under stress and RNA synthesis inhibitors treatments. Error bars represent mean±s.d. (n=25 cells from three independent biological replicates).

FIG. 8 is a chromatographic traces of LCMS/MS scans, including of HeLa Flpin cells treated with ^{DEA}tC.

FIG. 9A is an HPLC chromatogram time course of tCo phosphorylation by recombinant UCK2 WT.

FIG. 9B is a graph showing quantification of tCo and tC RNA incorporation in HeLa cell lines stably expressing inducible UCK2 WT.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

References listed herein are incorporated by reference in their entirety as if fully set forth herein.

The metabolic incorporation of modified nucleosides into cellular RNA is a versatile approach to study RNA transcription and turnover. It has been reported that modified pyrimidine and purine nucleosides containing alkyne and azide functionality can be incorporated into cellular RNA through nucleotide salvage pathways, labeled with fluorescent dyes using bioorthogonal chemistry, and used to visualize RNA in intact cells and organisms. Thus, unlike RNA imaging strategies relying upon exogenous sequence tags or recruitment of RNA-binding proteins, the power of metabolic labeling lies in its simplicity, ease of use, and transcriptome-wide generality. While many azide/alkyne-modifications on nucleosides minimally perturb the native structure and are therefore readily accepted by the nucleotide salvage pathway, imaging of these metabolically incorporated bioorthogonal ribonucleosides has been performed largely in fixed cells or tissue sections. This is primarily due to reliance on Cu(I)-catalyzed azide-alkyne cycloaddition (CuAAC) for fluorescent dye labeling, which is incompatible with living cells, as well as the need to “wash out” excess unreacted dye used to drive the labeling reaction to completion. While these approaches are powerful, fixation and bioorthogonal labeling can introduce bias and therefore it is unknown whether these methods accurately capture a snapshot of all cellular RNAs. In a few instances, strain-promoted azide-alkyne cycloaddition (SPAAC) or “Cu-free” click chemistry has been utilized to label azido-modified RNA with fluorescent dyes in living cells, however the slow kinetics of this reaction and difficulty in removing excess fluorophore make challenging its application to intracellular targets.

An alternative to the two-step procedure used for imaging RNA with bioorthogonal ribonucleosides would be the direct metabolic incorporation of fluorescent nucleosides. Since this strategy would not require a fluorophore labeling reaction after RNA incorporation and the fluorescence of an RNA transcript containing multiple modified nucleosides should exceed the fluorescence background generated by individual modified nucleosides or nucleotides, it should enable live-cell RNA imaging of global transcriptome dynamics. Indeed, a large number of fluorescent nucleoside analogs have been reported, however these structure have been primarily used for in vitro applications and incorporated into RNA use chemical synthesis or in vitro transcription, and it is unknown whether they are suitable substrates for metabolic incorporation into RNA.

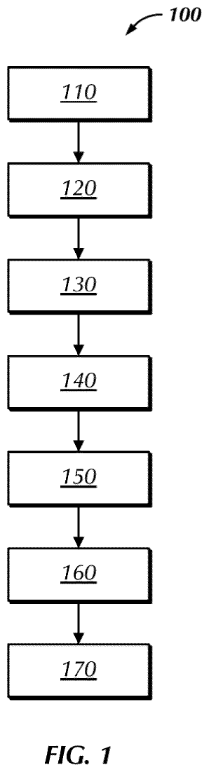

In one aspect of the present disclosure, a method for incorporating a fluorescent nucleoside into cellular RNA may be provided. Referring to FIG. 1,

The method may include providing 110 a fluorescent nucleoside. Any appropriate fluorescent nucleoside may be utilized here. In some embodiments, the fluorescent nucleoside may include an extended ring systems projecting from a non-Watson Crick face of the fluorescent nucleoside. Preferably, the nucleoside will be compatible with cellular metabolic enzymes and RNA polymerase enzymes. Therefore, the nucleosides are preferably of similar size to the canonical nucleosides (i.e. adenosine, uridine, cytidine, guanosine) and retain the ability to engage in Watson-Crick base pairing. The nucleosides must be soluble and cell permeable. For fluorescence imaging, nucleosides with higher molar extinction coefficients and quantum yield are preferred. Further, it is preferred that the nucleosides be compatible with common lasers and filters used on commercial confocal microscopes.

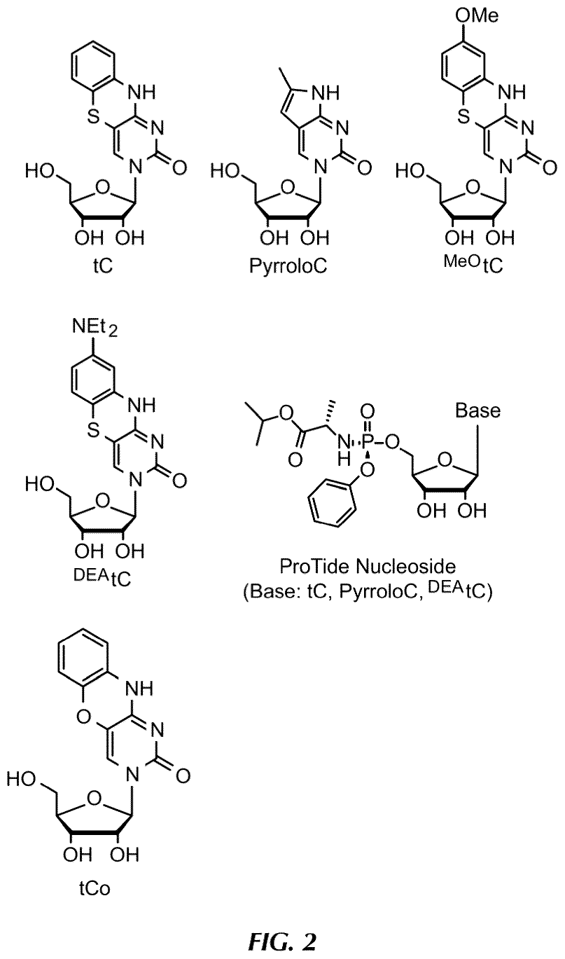

In some embodiments, the fluorescent nucleoside may be a fluorescent bicyclic and tricyclic cytidine analogue. In some embodiments, the fluorescent nucleoside may be a pyrimidine nucleoside. In some embodiments, the fluorescent nucleoside may be a C5-modified pyrimidine nucleoside. Some nucleosides that have been used for various examples disclosed herein can be seen in FIG. 2, including tC, PyrroloC, ^{MeO}tC, ^{DEA}tC, a ProTide nucleoside where “Base” is tC, PyrroloC, or ^{DEA}tC, and tCo. Each has a different absorbance and emission spectra that can be detected in use.

In some embodiments, the fluorescent nucleoside may have a structure according to structure (I), below:

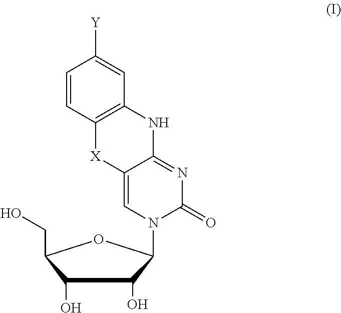

where X is S or O, and Y is H, NR₂, or OR, where R is a C1-C3 alkyl group.

In some embodiments, the fluorescent nucleosides may be commercially available. In some embodiments, providing the nucleoside may include chemically synthesizing the fluorescent nucleoside.

In one example, Starting materials and reagents were synthesized using reported procedures (^{DEA}tC, nucleobase, tC nucleobase, ^{MeO}tC nucleobase) or purchased at ACS reagent grade or higher from Acros Organic, Sigma-Aldrich, Combi-Blocks, and Berry & Associates, and used without further purification.

The method may include providing 120 a plurality of live cells that have been engineered, and specifically, configured to overexpress an UCK2 enzyme under control of an inducible promoter, a constitutively active promoter, or both.

In various embodiments, the live cells may be from any appropriate species. In some embodiments, human cells may be used, although other species, such as other mammals, reptiles, amphibians, or aves. In some embodiments, the cell may be a yeast or bacteria cell.

In various embodiments, the UCK2 enzyme may be such a WT or mutant ribonucleoside kinase UCK2. In some embodiments, the gene configured to express the mutant may have at least a 95%, 96%, 97%, 98%, or 99% homology to a WT UCK2 gene [SEQ ID NO. 1]. In some embodiments, the mutant has only a single substitution mutation, such as a Y65G mutant of UCK2.

In various embodiments, the inducible promoter may be any appropriate inducible promoter. In some embodiments, the promoter may be a light-inducible promoter (e.g., P_{C120}, which can be activated by, e.g., a light-sensitive VP16-EL222 protein), or a chemically-inducible promoter. Any appropriate light-inducible or chemically-inducible promoter may be used. In some embodiments, the chemically-inducible promoter may be, e.g., a tetracycline-inducible promoter.

The method may also include inducing 130 the inducible promoter to overexpress the WT or mutant ribonucleoside kinase UCK2. This may be accomplished by, e.g., exposing the cells to the inducing agent for an appropriate amount of time. In some examples disclosed herein, for nucleoside labeling experiments in Flp-In Hela cells overexpressing UCK2, HeLa cells were seeded at 0.4×10⁶ cells per well in 35 mm glass bottom petri dishes. After 16 hours, cells were induced for 16 hours with 1 μg/mL tetracycline.

In some embodiments, UCK2 was expressed using plasmid-based expression systems (e.g., via transient transfection). Methods of transient transfection are known in the art. Plasmid-based expression is typically characterized by plasmid copy numbers up to several hundred per cell. Expression plasmids usually carry the gene of interest under the control of a promoter, an origin of replication, and a marker gene for selection of plasmid-carrying clones. In addition, coding or non-coding or non-functional backbone sequences are frequently present on said plasmids (i.e., vectors). The presence of plasmids and the corresponding replication mechanism alter the metabolism of the host cell.

In some embodiments, the method may include exposing 140 the plurality of live cells to a first quantity of the fluorescent nucleoside for a first period of time. The first period of time is typically 1-24 hours, and may be, e.g., 6-12 hours. In some examples disclosed herein, after being exposed to tetracycline, the HeLa cells were treated with 500 μM of fluorescent nucleosides for 12 hours.

In some embodiments, the method may also include washing out 150 free nucleoside after the first period of time. In some embodiments, the cells may be washed a plurality of times. In some embodiments, at least one wash may include washing the cells using a balanced salt solution, such as Dulbecco's phosphate-buffered saline (DPBS). In some embodiments, at least one wash may include washing the cells using a basal medium for supporting growth of the cell, such as Dulbecco's Modified Eagle Medium (DMEM).

The method may also include exposing 160 the plurality of live cells to a second quantity of the fluorescent nucleoside for a second period of time. The first period of time is typically 1-24 hours, and may be, e.g., 6-12 hours.

The method may also include imaging 170 the plurality of live cells using epifluorescence microscopy.

### EXAMPLES

Herein, the synthesis of a series of fluorescent cytidine analogues are disclosed and their in vitro phosphorylation by UCK2 and metabolic incorporation into cellular RNA are evaluated. The expression of UCK2 in cells facilitates efficient RNA labeling with pyrroloC and tC fluorescent nucleosides. Further, the UCK2 system combined with tC feeding and confocal fluorescence microscopy are used to measure RNA transcription, turnover, and trafficking in live cells during normal and stress conditions. The effects of oxidative stress on RNA metabolism are revealed and the formation of a novel stress-dependent RNA-protein granule is identified. Thus, the disclosed approach provides a general approach for live-cell RNA imaging and reveals the spatiotemporal dynamics of cellular RNA during the oxidative stress response.

A strategy is developed for whole transcriptome RNA imaging in living cells. It is shown through quantitative confocal fluorescence microscopy and nucleoside LC-QQQ-MS that overexpression of UCK2 enables metabolic RNA labeling and direct fluorescence imaging with fluorescent ribonucleoside cytidine analogs tC and pyrroloC. Compared to prior approaches that typically rely upon metabolic labeling with azide- or alkyne-modified nucleosides followed by bioorthogonal conjugation with a suitable reactive fluorophore, this strategy is operationally simpler, compatible with live-cell imaging, and less prone to artifacts resulting from cellular fixation and/or permeabilization and washing conditions.

tC RNA labeling and confocal imaging are applied in order to study the dynamics of RNA synthesis and turnover during the oxidative stress response. The results show a dramatic and rapid increase in RNA turnover during sodium arsenite treatment, with a ˜50% reduction in bulk RNA transcript levels transcribed before stress induction after only 30 minutes. In contrast, RNA synthesis rates appear largely unchanged in this initial period but then decline after more prolonged exposure to sodium arsenite. These RNA-specific effects likely play an important role in reshaping gene expression programs during the integrated stress response, both by downregulating the abundance of individual mRNA transcripts and by affecting rRNA levels and ribosome availability. In addition, the formation of cytoplasmic arsenite-induced RNA granules that colocalize with the RNA helicase DDX6 but do not colocalize with the stress granule marker G3BP1 or the P-body protein Dcp1a are identified. Since these granules were only formed when tC RNA labeling was performed in the presence of NaAsO₂ and not during a NaAsO₂ chase, it is proposed that they contain RNA transcripts that were synthesized during stress conditions. Further, it is found that granule formation is sensitive to the RNA Pol I inhibitor ActD but largely insensitive to the RNA Pol II inhibitor α-amanitin, suggesting that granules contain rRNA. It is speculated that the accumulation of rRNA in cytoplasmic granules distinct from stress granules and P-bodies may be related to the elimination of misprocessed forms generated during oxidative stress. DDX6, as a human RNA helicase, plays crucial roles in RNA degradation. Despite high abundance in P-body, DDX6 forms multiple complexes outside of P-body.

The techniques in this disclosure demonstrate that fluorescent nucleosides can be incorporated into RNA through metabolic labeling and used to track RNA metabolism and intracellular trafficking. The key to this technology is judicious selection of nucleoside structure and overexpression of the ribonucleoside kinase UCK2. The bicyclic and tricyclic cytidine analogs explored herein likely represent a privileged scaffold for RNA metabolic labeling, and investigation of this class of modified nucleosides together with UCK2 engineering should lead to the development of fluorescent nucleosides with enhanced and varied spectral properties and context-dependent fluorescence. Beyond tracking localization and metabolism, analogous approaches could be utilized to incorporate diversely functionalized nucleoside analogs as biophysical probes to explore nucleic acid structure and protein-RNA interactions in their native context.

### Fluorescent Nucleosides for RNA Imaging

In order to label cellular RNA for biological imaging, a set of fluorescent pyrimidine nucleosides was selected and evaluated. The criteria included commercial availability or synthetic accessibility of the structure, ability of the modified nucleoside to engage in canonical Watson-Crick base pairing, likelihood of compatibility with pyrimidine salvage pathways, and fluorophore brightness and spectral overlap with commonly used excitation and emission wavelengths in fluorescence microscopy. Therefore, four modestly sized bicyclic and tricyclic fluorescent cytidine analogues containing extended ring systems projecting from the non-Watson Crick face of the molecule were chosen.

PyrroloC is a commercially available bicyclic cytidine analogue with ε_{8350 nm}=5900 M⁻¹ cm⁻¹ and Φ_{em,460 nm} estimated at 0.2. It has the smallest structural perturbation among this set, but is significantly quenched by base pairing and stacking, with Φ_{em,460 nm}=0.06 in duplex DNA or RNA. The remaining three compounds are members of the tC family. Parent tC offers robust fluorescence both as a free nucleoside ε_{377 nm}=4700 M⁻¹ cm⁻¹ and Φ_{em,513 nm}=0.09 in 1× PBS buffer at pH 7.4, with slightly enhanced brightness in matched duplex DNA, Φ_{em,513 nm}=0.11. An extended tC family was synthesized and studied, finding that ^{DEA}tC has little fluorescence as a free nucleoside (Φ_{em,493 nm}=0.006) but much higher quantum yields of Φ_{em,500 nm}=0.12 in dsDNA or greater in a DNA-RNA heteroduplex. ^{MeO}tC lands in the middle, modestly brighter than ^{DEA}tC as a free nucleoside (Φ_{em,550 nm}=0.01) but with less fluorescence increase upon matched base pairing and stacking in dsDNA (Φ_{em,532 nm}≈0.03). The brightness of parent tC in all known biomolecular contexts is attractive, while ^{DEA}tC's fluorescence turn-on properties offer potential utility for probing nucleic acid metabolism in cells.

Metabolic Labeling with Fluorescent Cytidine Analogues.

It was next evaluated whether the fluorescent nucleosides could be incorporated into living cells. For this purpose, HeLa cells expressing ribonucleoside kinase UCK2 under the control of a tetracycline-inducible promoter (i.e., Flp-In T-Rex system) were used. The incorporate was monitored using fluorescence microscopy and quantitative nucleoside LC-QQQ-MS. The T-Rex system provides a convenient method to analyze the effect of UCK2 expression on nucleoside incorporation. Previously, it has been shown that UCK2 is rate limiting for the metabolic incorporation of modified pyrimidines through salvage pathways. Further, a Y65G mutant version of UCK2 was previously identified that exhibits enhanced activity on C5-modified pyrimidine nucleosides and can direct their incorporation into cellular RNA.

Here, overexpression of WT or mutant UCK2 protein is applied to facilitate the phosphorylation and incorporate fluorescent nucleosides, in the case that these modified structures are incompatible with native metabolism.

To test nucleoside incorporation, HeLa cells were treated with 500 μM of each fluorescent nucleoside for 12 hr and then imaged cells by epifluorescence microscopy after washing out free nucleoside. A DAPI filter set (375 nm, 435 nm) was chosen due to its availability on most fluorescence microscopes and since the excitation and emission band pass wavelengths overlapped with the spectra of each of the synthetic nucleosides, although the degree of overlap varies considerably based upon the nucleoside structure. Further, it was tested whether induction of UCK2 expression (WT or Y65G mutant) affects incorporation levels. Gratifyingly, in cells treated with tC or pyrroloC and overexpressing WT or Y65G UCK2, strong fluorescence labeling was observed. Fluorescence signal was concentrated in the nucleus, but was also found in the cytoplasm, consistent with RNA incorporation. Interestingly, for both pyrroloC and tC, we observed higher fluorescence in cells overexpressing WT UCK2 as compared to the Y65G mutant, in contrast to previous results with C5-modified uridine analogues with linear substitutions. In cells without UCK2 overexpression, minimal fluorescence (52-fold less) was detected, indicating that endogenous UCK2 levels are insufficient for robust incorporation of these fluorescence nucleosides. For the ^{MeO}tC and ^{DEA}tC derivatives, fluorescent cellular labeling was not observed under any of the conditions tested. In addition, ^{DEA}tC displayed significant cytotoxicity.

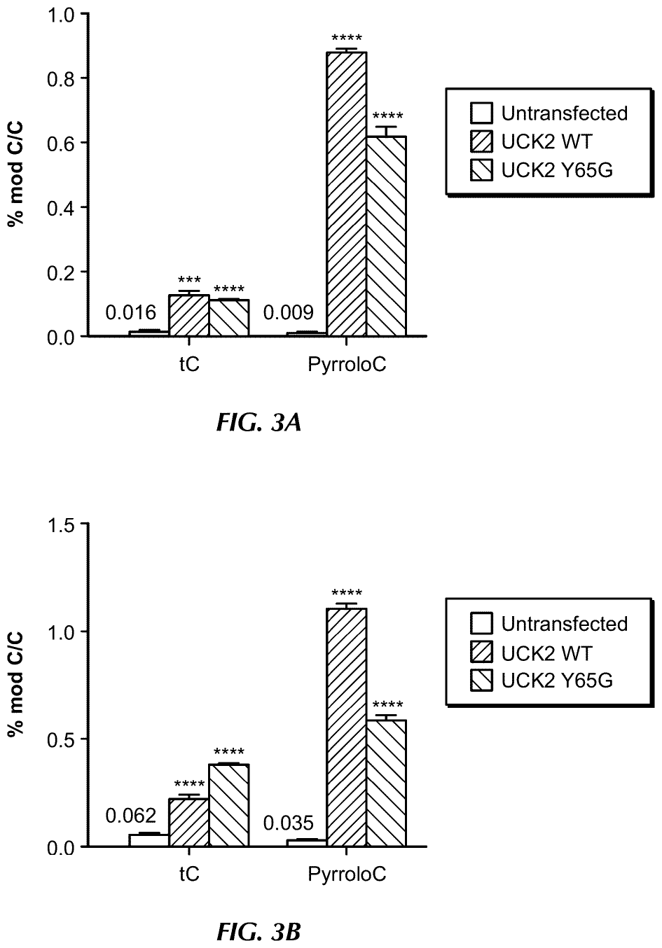

To prove that cellular fluorescence is a result of RNA labeling with pyrroloC and tC, rather than just uptake and phosphorylation of the nucleoside, two different assays were employed. First, feeding was performed in the presence of actinomycin D (ActD), a potent inhibitor of RNA polymerase I and II. Under these conditions, cellular fluorescence was greatly reduced (15-fold less). In contrast, co-treatment with hydroxurea (a DNA synthesis inhibitor) had little effect on labeling. In addition, total cellular RNA was isolated and nucleoside LC-QQQ-MS was performed after digestion and dephosphorylation to nucleosides. Strikingly, the results show that overexpression of WT or Y65G UCK2 kinase leads to ˜20-100-fold increase in RNA incorporation of pyrroloC. See FIGS. 3A and 3B. The highest level of pyrroloC labeling (1.1±0.02% of C) was achieved using cells expressing WT UCK2. See FIG. 3B. Similarly, a large increase in RNA labeling with tC was observed in both WT UCK2 and Y65G UCK2 cells, although the overall levels of incorporation were lower than those achieved with pyrroloC nucleoside. See FIGS. 3A and 3B. Incorporation of ^{DEA}tC and ^{MeO}tC was also measured using the LC-QQQ-MS assay. ^{DEA}tC nucleoside was unable to be detected in digested total RNA, consistent with cellular imaging experiments. Further, while 0.026±0.0009% incorporation of ^{MeO}tC was measured upon UCK2 induction, this level is ˜10-40-fold lower than with pyrroloC or tC. Taken together, the data demonstrates that metabolic RNA labeling with fluorescent nucleosides pyrroloC and tC can be achieved through overexpression of nucleoside kinase UCK2. This system should be suitable for live-cell imaging of the bulk RNA population.

In Vitro Phosphorylation with UCK2

To further understand the incorporation of bulky fluorescent nucleosides into cellular RNA and the involvement of UCK2 in this process, the phosphorylation of modified nucleosides by recombinant, purified UCK2 was studied using an HPLC-based in vitro assay. While it is presumed that UCK2 phosphorylation is required for metabolic incorporation, the lack of incorporation of the ^{DEA}tC and ^{MeO}tC derivatives could be a result of failure of downstream metabolic steps rather than poor UCK2 phosphorylation. Consistent with their robust cellular incorporation, efficient phosphorylation of pyrroloC and tC nucleosides by UCK2 was observed, with complete phosphorylation of pyrroloC and 50% phosphorylation of tC in 30 minutes under the conditions assayed. In contrast, no phosphorylation of ^{DEA}tC (even after extended reaction times) was detected and only minimal phosphorylation (<20%) of ^{MeO}tC after 1 hr. The results show that the incorporation efficiency of bicyclic and tricylic fluorescent cytidine analogues into cellular RNA correlates strongly with their ability to be phosphorylated by UCK2. Interestingly, despite low level in vitro phosphorylation of ^{MeO}tC by UCK2, no incorporation could be detected of this nucleoside into cellular RNA, suggesting that downstream metabolism of ^{MeO}tC-derived nucleotides may also be compromised.

In previous work, it was found the modest substitutions at the C5 position of pyrimidine nucleosides severely compromised the ability of uridine nucleosides to serve as substrates for UCK2, likely due to steric clash with aromatic residues in the catalytic center of the enzyme (i.e., Y65). Therefore, it was surprising that pyrroloC and tC were efficient substrates for UCK2 due to their large polycyclic structures. Since previously only C5-modified uridine nucleosides had been studied, the example results were compared with pyrroloC and tC against two cytidine analogues containing different size substitutions at the C5 position: 5-bromocytidine (5-BrCyd) and 5-iodocytidine (5-ICyd). As expected, UCK2 activity on these substrates was inversely related to the size of the C5 substituent.

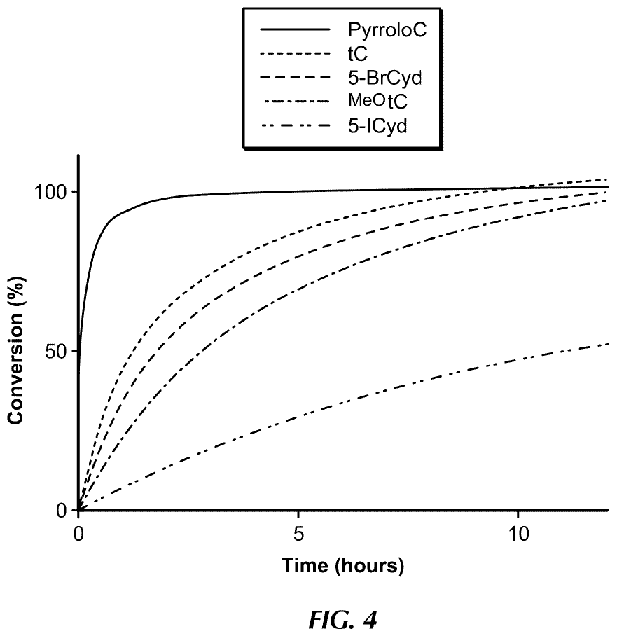

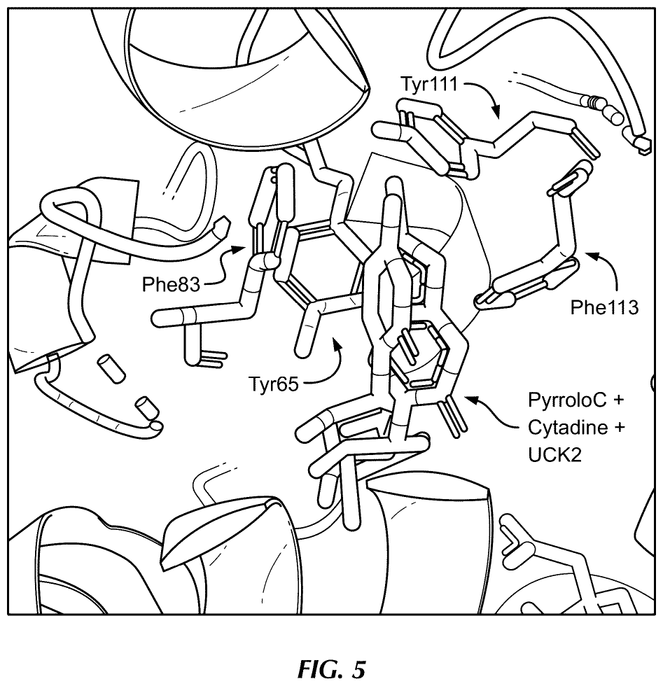

Surprisingly, pyrroloC and tC were both more efficient UCK2 substrates than 5-BrCyd (see FIG. 4), despite their multicyclic structure. It is hypothesized that the rigidity of the pyrroloC and tC structures presents less steric bulk in proximity to UCK2 Y65, and indeed docking studies show that both nucleosides are well accommodated in the UCK2 active site. In addition, the extended pi-systems presented by pyrroloC and tC could engage in productive stacking or Van der Waals interactions with aromatic residues in the UCK2 active site, such as Phe83, Tyr65, Phe113, or Tyr111. See FIG. 5.

ProTide Fluorescent Nucleoside Derivatives are Poorly Incorporated into RNA

Due to the reliance of fluorescent nucleoside incorporation on UCK2 activity, it was also decided to explore kinase bypass methods as a strategy to facilitate metabolic labeling. These approaches, which typically involve masked phosphate groups, allow nucleotides to be delivered across the plasma membrane and rely on subsequent enzymatic unmasking in the cytoplasm. One of the most successful of these approaches is the ProTide technology pioneered by McGuigan and used clinically in drugs including sofosbuvir and tenefovir alafenamide. ProTide versions of the fluorescent nucleosides could provide an alternative approach to incorporate these structures into nascent RNA without the need for UCK2 overexpress. Therefore, ProTide derivatives of pyrroloC, tC, and ^{DEA}tC were prepared, and their uptake into cells and incorporation into cellular RNA was investigated using fluorescence microscopy and LC-QQQ-MS. Of the three ProTide compounds assayed, cellular fluorescence was only detected in cells treated with ProTide-tC, suggesting that ProTide-pyrroloC and ProTide-^{DEA}tC failed to penetrate cells, or were rapidly washed out before imaging. Strangely, fluorescence resulting from ProTide-tC treatment was restricted to the cytoplasm and exhibited punctate staining, which is inconsistent with RNA labeling. To further confirm that ProTide-tC was not incorporated into RNA, cells were co-treated with ActD and no reduction in signal was found. Similarly, LC-QQQ-MS of isolated RNA from ProTide-tC treated cells indicated minimal incorporation-lower than that of cells treated with tC nucleoside. While it does appear that ProTide-tC can diffuse across the plasma membrane, it is likely that unmasking is inefficient in Hela cells, and further the compound appears to concentrate in cellular vesicles, precluding the use of the ProTide strategy for RNA metabolic labeling using tC and pyrroloC-derived nucleosides in this system.

### Live Cell RNA Imaging

Efficient cellular RNA labeling with tC and pyrroloC upon UCK2 expression led us to pursue this system for imaging real-time RNA dynamics in living cells. Towards this goal, UCK2-expressing Hela cells were treated with pyrroloC or tC and confocal fluorescence microscopy was used to detect labeled RNA. First, compatibility with different excitation sources and filter sets was explored. For confocal imaging, 405 nm excitation combined with a DAPI emission filter gives similar signal intensity for both pyrroloC and tC; however, tC can also be imaged using 405 nm excitation combined with a GFP emission filter due to its longer wavelength emissions, and indeed this combination gives better signal and allows one to reduce exposure time in half (e.g., from 1 second exposure time to 0.4 second exposure time).

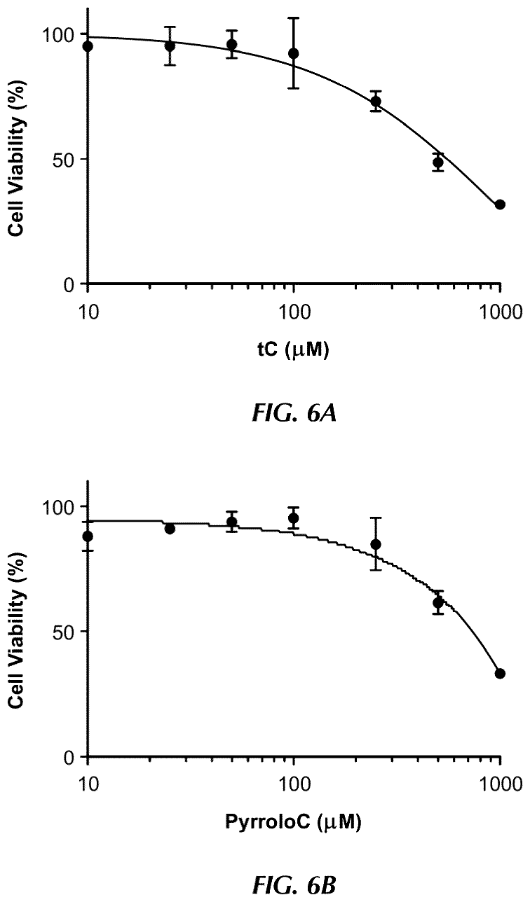

Therefore, it was decided to use the tC/UCK2 pair for confocal imaging with 405 nm excitation combined with GFP emission filter. First, RNA labeling with different serum concentrations and in the presence of the RNA synthesis inhibitor actinomycin D (ActD) were evaluated. Cells were treated with 200 μM tC, a concentration that balances cellular toxicity with incorporation efficiency (see FIGS. 6A, 6B) and fluorescence was monitored after 6 hr.

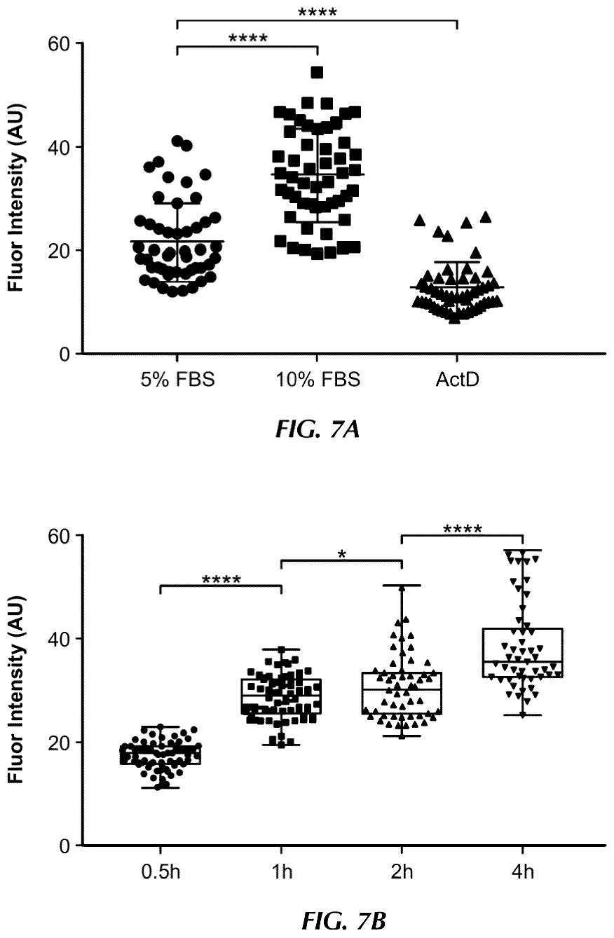

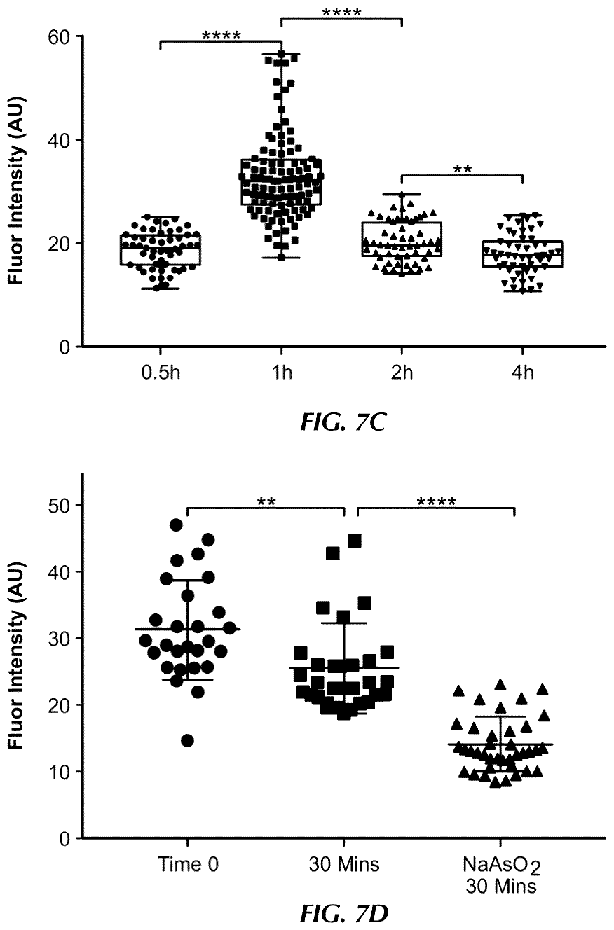

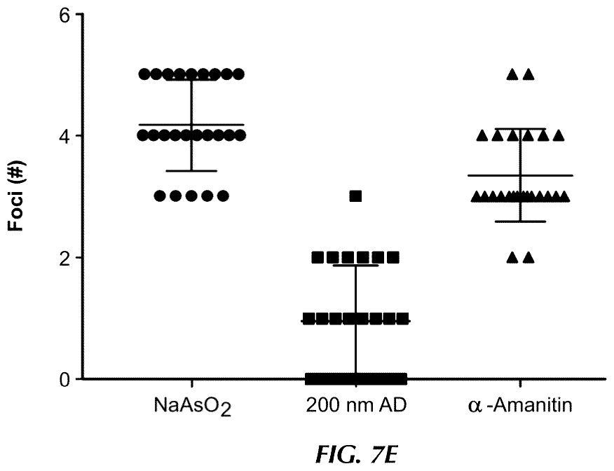

tC fluorescence was observed in the cytoplasm and nucleus with enhanced signal at the nucleolus, the major site of rRNA synthesis, consistent with incorporation into RNA transcripts. Fluorescence was decreased substantially with ActD co-treatment, indicating that the majority of signal originates from tC-containing RNA transcripts as opposed to fluorescent tC metabolites (nucleotides). In addition, a 1.6 fold increase in fluorescence signal was found when tC treatment was performed in the presence of 10% fetal bovine serum as compared to reduced (e.g., 5%) serum concentrations (see FIG. 7A).

Next, the kinetics of RNA labeling with tC were investigated. Cellular fluorescence was observed at time points as early as 30 min (see FIG. 7B), indicating that tC RNA labeling can be used to report on dynamic cellular processes. Fluorescence signal increased over time and reached a plateau at 6 hr.

Next, tC RNA labeling was used to investigate global RNA synthesis and turnover during oxidative stress. Oxidative stress induces a number of changes in cellular physiology concomitant with alterations in translational and transcriptional programs, but the impact of oxidation on bulk RNA dynamics is still poorly understand. To investigate RNA dynamics during stress conditions metabolic labeling was performed with tC nucleoside during sodium arsenite-induced oxidative stress and imaged cells at multiple time points after initiating feeding and stress. A biphasic response during arsenite stress was observed-tC fluorescent increased during the first hour of treatment but then steadily decreased from 1 hr to 4 hr of continuous arsenite exposure (see FIG. 7C). In contrast, tC accumulation in RNA in unstressed cells continuously increases during the course of 4 hr (see FIG. 7B). In addition, NaAsO2-treated cells displayed changes in nucleolar RNA signal, exhibiting larger numbers of nucleoli with reduced size and more regular spherical shape as compared to unstressed cells. Since NaAsO2 exposure could affect both RNA synthesis and degradation, a pulse-chase labeling experiment was next performed in order to specifically investigate RNA turnover. Cells were fed tC for 4 hr followed by a 30 min chase in tC-free medium in the presence or absence of NaAsO2. Fluorescence signal from tC-labeled RNA was drastically reduced (2.2-fold reduction) during chase in AsO2-containing medium whereas tC-labeled RNA levels were largely unperturbed (23% decrease) in unstressed cells during the 30 min chase. In FIG. 7D, time 0 is the start of the 30 min chase. Taken together, this shows that bulk RNA degradation rapidly increases in response to NaAsO2 stress, but they do not support an acute effect of arsenite stress on RNA synthesis. In addition, it is altered nucleolar RNA localization is found, which is suggestive of oxidative stress effecting rRNA synthesis and/or processing. Interestingly, further analysis of RNA localization upon NaAsO2 treatment indicated the formation of cytoplasmic RNA foci that arose as early as 1 hr after induction of stress. These foci were only present when NaAsO₂ stress was applied during tC feeding, and were absent from unstressed cells or cells where NaAsO₂ was applied during the chase (i.e., after tC treatment).

To categorize the observed foci, the foci were compared against known cytoplasmic RNA-based structures specific to oxidative stress conditions. Stress granules are RNA-protein condensates containing translationally stalled mRNA and RNA binding proteins that form rapidly after arsenite stress. It was tested whether the observed foci colocalized with the characteristic stress-granule protein G3BP1 using live-cell imaging and expression of G3BP1-mCherry prior to tC labeling and NaAsO₂ treatment. As expected, G3BP1-mCherry exhibited diffuse cytosolic staining during normal growth conditions and rapidly formed micronized foci in the cytosol upon NaAsO₂ stress, but these foci did not colocalize with the tC-labeled RNA foci, although they were frequently found in close proximity.

Next, co-localization with the P-body protein Dep1a was investigated. While P-bodies are present constitutively, the number of P-bodies has been shown to increase upon NaAsO2 stress. Dcp1a-mCherry was imaged together with tC-labeled RNA, but colocalization was not observed between the NaAsO₂-dependent RNA foci and P-bodies. Finally, tC RNA live cell imaging was combined with expression of DDX6-mCherry and it was found that tC RNA foci consistently co-localized with DDX6 upon NaAsO₂ stress.

Further, to investigate the nature of the RNA in these foci, their sensitivity to different small-molecule RNA polymerase inhibitors was tested. See FIG. 7E. Foci formation was unaffected by alpha-amanitin, an RNA polymerase II inhibitor, but abolished with ActD treatment at concentrations specific for RNA polymerase I inhibition. Taken together, our results show that a subset of RNA transcripts generated during arsenite stress accumulate in cytoplasmic foci together with the RNA helicase DDX6. These foci are distinct from canonical stress granules and P-bodies and likely contain rRNA.

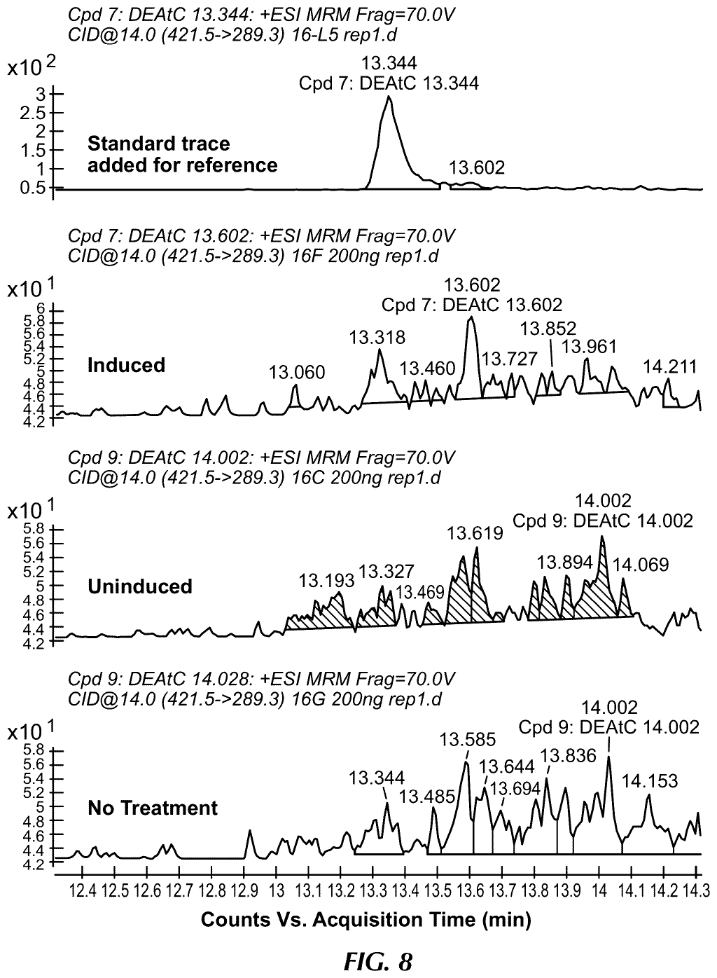

Separately, it is noted that as seen in FIG. 8, incorporation of ^{DEA}tC can be detected via, e.g., a chromatic trace using LCMS/MS. In FIG. 8, induced and uninduced HeLa Flpin UCK2 cells were treated with ^{DEA}tC, and were then analyzed via LCMS/MS.

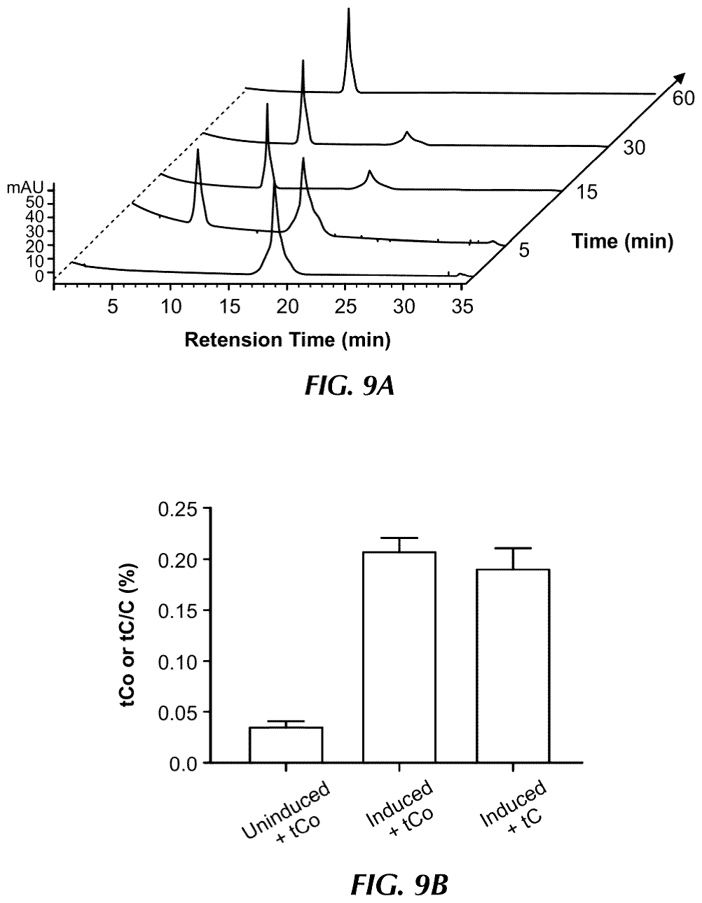

Separately, it is noted that other bicyclic and tricyclic fluorescent nucleosides can be incorporated into cellular RNA. FIG. 9A is an HPLC chromatogram time course of tCo phosphorylation by recombinant UCK2 WT, illustrating showing the detection of tCo incorporation into cellular RNA through UCK WT overexpression. tCo's structure can be seen in FIG. 2. In FIG. 9B, one can see quantification of tCo and tC RNA incorporation in HeLa cell lines stably expressing inducible UCK2 WT. Images were captured of metabolic incorporation of tCo into cellular RNA in the presence of 2 μM ActD or 10 mM hydroxyurea. Similar to the results for tC and PyrroloC disclosed herein, cellular fluorescence was greatly reduced in the presence of ActD, while co-treatment with hydroxyurea had little effect on fluorescence.

In another aspect, a kit may be provided. The kit may include a plurality of cells as disclosed herein, configured to overexpress a WT or mutant ribonucleoside kinase UCK2 under control of an inducible promoter, a constitutively active promoter, or both. The kit may include a fluorescent nucleoside as disclosed herein. The kit may optionally include a chemical agent adapted to induce the inducible promoter.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

SEQUENCES

SEQ ID NO. 1 (UCK2)
MAGDSEQTLQ NHQQPNGGEP FLIGVSGGTA SGKSSVCAKI
VQLLGQNEVD YRQKQVVILS QDSFYRVLTS EQKAKALKGQ
FNFDHPDAFD NELILKTLKE ITEGKTVQIP VYDFVSHSRK
EETVTVYPAD VVLFEGILAF YSQEVRDLFQ MKLFVDTDAD
TRLSRRVLRD ISERGRDLEQ ILSQYITFVK PAFEEFCLPT
KKYADVIIPR GADNLVAINL IVQHIQDILN GGPSKRQTNG
CLNGYTPSRK RQASESSSRP H

What is claimed is:

1. A method for incorporating a fluorescent nucleoside into cellular RNA, comprising:
exposing a plurality of live cells configured to overexpress a WT or mutant ribonucleoside kinase UCK2 under control of an inducible promoter, a constitutively active promoter, or both, to a first quantity of a fluorescent nucleoside for a first period of time.

2. The method according to claim 1, further comprising chemically synthesizing the fluorescent nucleoside.

3. The method according to claim 1, wherein the fluorescent nucleoside comprises an extended ring systems projecting from a non-Watson Crick face of the fluorescent nucleoside.

4. The method according to claim 3, wherein the fluorescent nucleoside is a fluorescent bicyclic and tricyclic cytidine analogue.

5. The method according to claim 1, further comprising washing out free nucleoside after the first period of time.

6. The method according to claim 1, further comprising inducing the inducible promoter to overexpress the WT or mutant ribonucleoside kinase UCK2.

7. The method according to claim 6, wherein the inducible promoter is a chemically inducible promoter.

8. The method according to claim 7, wherein the chemically inducible promoter is a tetracycline-inducible promoter.

9. The method according to claim 6, further comprising exposing the plurality of live cells to a second quantity of the fluorescent nucleoside for a second period of time.

10. The method according to claim 1, wherein the WT or mutant ribonucleoside kinase UCK2 is a Y65G mutant of UCK2.

11. The method according to claim 1, wherein the plurality of live cells are human cells.

12. The method according to claim 1, further comprising imaging the plurality of live cells using epifluorescence microscopy.

13. A kit, comprising:
a plurality of cells configured to overexpress a WT or mutant ribonucleoside kinase UCK2 under control of an inducible promoter, a constitutively active promoter, or both;
a fluorescent nucleoside; and
optionally, a chemical agent adapted to induce the inducible promoter.
